(19) United States
(12) Patent Application Publication
(10) Pub. No.: US 2026/0021304 A1
(43) Pub. Date: Jan. 22, 2026

(54) NERVE STIMULATION SYSTEM

(71) Applicant: Covidien LP, Mansfield, MA (US)

(72) Inventors: Randal C. Schulhauser, Phoenix, AZ (US); Ashwini D. Sharan, Pennington, NJ (US); Merdim Sonmez, Irvine, CA (US); Tarek D. Haddad, Minneapolis, MN (US); Darrell J. Swenson, Lino Lakes, MN (US); Walton W. Baxter, San Clemente, CA (US); Daniel J. Raible, Arvada, CO (US); Mark P. Ashby, Laguna Niguel, CA (US); Scott R. Stanslaski, Shoreview, MN (US); Gary P. Kivi, Maple Grove, MN (US); Gautham Ravindran, Lino Lakes, MN (US); Benjamin P. Isaacson, Mahtomedi, MN (US)

(21) Appl. No.: 19/254,450

(22) Filed: Jun. 30, 2025

Related U.S. Application Data
(60) Provisional application No. 63/674,131, filed on Jul. 22, 2024.

(51) Int. Cl. A61N 1/36; A61N 1/02; A61N 1/05; A61N 1/372

(52) U.S. Cl. CPC A61N 1/36135; A61N 1/025; A61N 1/36053; A61N 1/36103; A61N 1/36125; A61N 1/37288; A61N 1/0526

(57) ABSTRACT

An example system includes a first implantable medical device (IMD) configured to sense electroencephalogram (EEG) signals of a patient; a second IMD configured to determine evoked compound action potential (ECAP) of the patient; and processing circuitry of at least one of the first IMD, the second IMD, or a computing device configured for wireless communication with at least one of the first IMD or the second IMD, the processing circuitry configured to determine a nerve tissue stimulation command based on at least one of the EEG signals and the ECAP, wherein the second IMD is further configured to apply an electrical signal to stimulate nerve tissue based on the nerve tissue stimulation command.

### CROSS-REFERENCE TO RELATED APPLICATIONS

This application claims the benefit of and priority to U.S. Provisional Patent Application Ser. No. 63/674,131 filed Jul. 22, 2024, the entire disclosure of which is incorporated by reference herein.

### TECHNICAL FIELD

The disclosure relates to devices and techniques for a nerve stimulation system.

### BACKGROUND

Neuromodulation by electrical stimulation of nerves, such as the vagus nerve, median nerve, renal nerve, splenic nerve, etc., has been shown to be useful for a wide range of purposes. Some proposed systems for vagus nerve stimulation include cuff electrodes. Implantation of cuff electrodes for vagus nerve stimulation typically involves a relatively invasive and complex surgical procedure, as well as negative tissue interactions. Consequently, some proposed systems for vagus nerve stimulation include vascular access leads.

### SUMMARY

Electrical stimulation of a nerve, such as a vagus nerve, may provide therapy in response to cranial acute health events, such as stroke. However, nerve stimulation not provided within a short time after a cranial acute health event and/or not applied at particular parameters may be ineffective. In general, the disclosure is directed to devices, systems, and techniques for providing personalized closed loop therapy by sensing particular patient parameters, such as particular biomarkers, and providing nerve tissue stimulation of a patient.

In some examples, the disclosure is directed to devices, systems, and techniques sensing an electroencephalogram (EEG) signal and an evoked compound action potential (ECAP), determining a nerve tissue stimulation command based on at least one of the EEG signal or the ECAP, and applying an electrical signal to stimulate nerve tissue, such as vagus nerve tissue, based on the received nerve tissue stimulation command.

In some examples, the disclosure is directed to devices, systems, and techniques for sensing an EEG signal during a first period of time, determining an initial indication of a cranial acute health event based on the EEG signal, and stimulating nerve tissue, such as median nerve tissue, based on a determination of the initial indications of a cranial acute health event. The devices, systems and techniques may further determine an evoked potential (EP) based on a sensed EP signal during a second period of time, the second period of time being after the first period of time and after the nerve tissue (e.g., median nerve tissue) is stimulated, determining/confirming the initial indication of the cranial acute health event is a cranial acute health event based, at least, one the EP satisfying a cranial acute health event EP threshold, and/or stimulating vagus nerve tissue in response to the determination that the initial indication of the cranial acute health event is a cranial acute health event.

In some examples, the techniques for performing personalized closed loop sensing and nerve tissue stimulation of a patient described herein may provide for more timely personalized nerve tissue stimulation therapy being applied timely in response to a cranial acute health event and/or being applied at a particular amplitude, frequency, and/or location that produces improved patient response rate to nerve tissue stimulation therapy. The improved patient response rate may reduce complications due to a cranial acute health event, such as, but not limited to, brain injury or death.

In one example, this disclosure describes a system comprising: a first implantable medical device (IMD) configured to sense electroencephalogram (EEG) signals of a patient; a second IMD configured to determine evoked compound action potential (ECAP) of the patient; and processing circuitry of at least one of the first IMD, the second IMD, or a computing device configured for wireless communication with at least one of the first IMD or the second IMD, the processing circuitry configured to determine a nerve tissue stimulation command based on at least one of the EEG signals and the ECAP, wherein the second IMD is further configured to apply an electrical signal to stimulate nerve tissue based on the nerve tissue stimulation command.

In another example, this disclosure describes a system comprising: a first implantable medical device (IMD) configured to sense electroencephalogram (EEG) signals of a patient and sense an evoked potential (EP) signal of the patient; a second IMD configured to an apply electrical signal to stimulate nerve tissue; and processing circuitry of at least one of the first IMD, the second IMD, or a computing device configured for wireless communication with at least one of the first IMD or the second IDM, the processing circuitry configured to: determine an initial indication of a cranial acute health event based on the EEG signals during the first period of time; and determine a nerve tissue stimulation command based on the determination of the initial indication of the cranial acute health event, wherein the second IMD is further configured to apply an electrical signal to stimulate nerve tissue based on the nerve tissue stimulation command, and wherein the processing circuitry is further configured to determine an EP occurring during a second period of time based on the sensed EP signal, the second period of time being after the first period of time and after the second IMD applies an electrical signal to stimulate nerve tissue, and determine the initial indication of the cranial acute health event is a cranial acute health event based, at least, on the EP satisfying a cranial acute health event EP threshold.

The details of one or more examples are set forth in the accompanying drawings and the description below. Other features, objects, and advantages of the disclosure will be apparent from the description and drawings, and from the claims.

The above summary is not intended to describe each illustrated example or every implementation of the present disclosure.

### BRIEF DESCRIPTION OF DRAWINGS

FIG. 1 depicts a conceptual diagram of an example neurostimulation system in accordance with one or more techniques of the present disclosure.

FIG. 2A is a conceptual diagram of an example sensor device in accordance with one or more techniques of the present disclosure.

FIG. 2B is a conceptual diagram of an example sensor device in accordance with one or more techniques of the present disclosure.

FIG. 3 depicts a conceptual diagram of an example of a lead in accordance with one or more techniques of the present disclosure.

FIG. 4 depicts a schematic diagram of an example of an implantable medical device in accordance with one or more techniques of the present disclosure.

FIG. 5 is a schematic diagram of an example stroke detection system configured in accordance with one or more techniques of the present disclosure.

FIG. 6A depicts a top view of an example sensor device in accordance with one or more techniques of the present disclosure.

FIG. 6B depicts a side view of the example sensor device shown in FIG. 6A in accordance with one or more techniques of the present disclosure.

FIG. 6C depicts a top view of an example sensor device in accordance with one or more techniques of the present disclosure.

FIG. 6D depicts a side view of an example sensor device in accordance with one or more techniques of the present disclosure.

FIG. 6E depicts a side view of an example sensor device in accordance with one or more techniques of the present disclosure.

FIG. 6F depicts a side view of an example sensor device in accordance with one or more techniques of the present disclosure.

FIG. 7 depicts an example sensor device in accordance with one or more techniques of the present disclosure.

FIG. 8 depicts an example target region for the sensor devices in accordance with one or more techniques of the present disclosure.

FIG. 9 is a diagram of an example map for electroencephalography (EEG) sensor measurements.

FIG. 10 is a flow diagram illustrating example techniques that may be performed by a one or more of a system or device, in accordance with one or more techniques disclosed herein.

FIG. 11 is a flow diagram illustrating example techniques that may be performed by a one or more of a system or device, in accordance with one or more techniques disclosed herein.

### DETAILED DESCRIPTION

Vagus nerve stimulation may assist in stroke rehabilitation and limit ischemia reperfusion injury. After a myocardial infarct or stroke, reperfusion therapies (surgery or drugs) are given to restore blood flow. However, due to the restoration of blood, flow induced local damage occurs, which is called ischemia reperfusion injury. This will induce local accumulations of chemical mediators such as reactive oxygen species (ROS) production, inflammatory cytokines, bradykinin, etc. Thus, the inflammatory state is worsened. The inflammatory compounds will trigger sensory signaling, which might lead to a reduced organ vagus activity and sympathetic overdrive. Vagus nerve stimulation may treat reperfusion damage as the inflammatory state may be lowered by increasing parasympathetic drive. In some examples, vagus nerve stimulation may be applied as treatment in response to a particular change in epileptic inter-ictal spiking.

In general, the disclosure is directed to devices, systems, and techniques for sensing particular patient parameters and performing closed loop nerve tissue stimulation of a patient. For example, stimulating vagus nerve tissue and/or median nerve tissue of the patient from one or more electrodes, such as one or more electrodes on a lead disposed within or beside a lumen of a patient, such as an internal jugular vein (IJV) of the patient. Sensing an EEG signal and an ECAP signal and determining and then applying nerve tissue stimulation, such as vagus nerve tissue stimulation, based on at least one of the sensed EEG signal and/or ECAP signal may provide quicker, more efficient, and more responsive nerve tissue stimulation than an open-loop technique.

In addition, determining an initial indication of a cranial acute health event based on a sensed EEG signal, stimulating nerve tissue, such as median nerve tissue, in response to the determination of the initial indication of a cranial acute health event, determining an EP based on a sensed EP signal, and determining/confirming the initial indication of the cranial acute health event is a cranial acute health event based on the EP satisfying a cranial acute health event EP threshold, may provide for improved detection of cranial acute health events, such as stroke, with reduced false positives. In addition, stimulating vagus nerve tissue in response to the determination/confirmation that the initial indication of the cranial acute health event is a cranial acute health event may provide quicker, more efficient, and more responsive results than an open-loop technique. This disclosure describes examples of such devices, systems, and techniques.

A device or system configured to stimulate nerve tissue, such as vagus nerve tissue and/or median nerve tissue, as described in examples above and below, would be useful for treating a variety of illnesses including, but not limited to: reperfusion damage, cardiac ischemia, brain ischemia, stroke, traumatic brain injury, and/or postoperative cognitive decline or postoperative delirium. Treating any of these diseases may improve patient outcomes by shortening length of hospital stays and reducing medical costs.

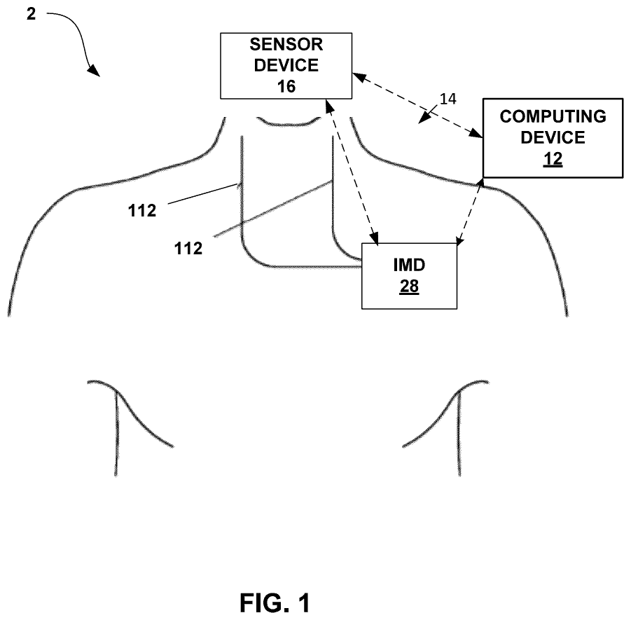

FIG. 1 is a conceptual diagram illustrating an example neurostimulation system 2. Neurostimulation system 2 includes an implantable medical device (IMD) 28, one or more leads 112 coupled to IMD 28, and sensor device 16. In some examples, sensor device 16 may be communicatively coupled to IMD 28 and/or computing device 12. In some examples, computing device 12 may be communicatively coupled to IMD 28 and/or sensor device 16. In some examples, IMD 28 may be communicatively coupled to computing device 12 and/or sensor device 16. In some examples, one or more leads 112 may each include one or more electrodes (not shown in FIG. 1) and be configured to provide stimulation signal(s) to one or more nerves, such as a vagus nerve or median nerve, of patient 14. In some examples, one or more sensors of sensor device 16, IMD 28, and/or lead 112 may be for sensing one or more of an electroencephalogram (EEG) signal, electrocardiogram (ECG) signal, respiration, blood pressure, activity level, electromyography (EMG) signal, evoked potential (EP) signal, evoked compound action potential (ECAP) signal, and/or electrophysiology, and may comprise electrodes, accelerometers, or any other known sensors. One or more sensors of sensor device 16 may additionally or alternatively include one or more sensors to identify changes in EEG signals, blood pressure, pulse, blood flow, respiration, temperature, or any metrics indicative of nerve tissue stimulation.

In some examples, IMD 28 may determine an ECAP of patient 14 based on ECAP signal(s), such as ECAP signal(s) sensed via one of more electrodes on lead 112. In some examples, IMD 28 may include one or more sensors, such as one or more electrodes, to sense ECAP signal(s). In some examples, the ECAP may represent the synchronous firing of a population of electrically stimulated nerve fibers, such as the synchronous firing of the corresponding respective nerve (e.g., vagus nerve or median nerve) that was stimulated by IMD 28. In some examples, neurostimulation system 2 may provide one or more stimulation pulses, for example a single stimulation pulse, via one or more electrodes and the ECAP may be recorded by another electrode or sensor to assess if nerve tissue stimulation takes place, such as if vagus nerve tissue stimulation takes place.

In some examples, one or more leads 112 may be configured to be positioned endovascularly with respect to a vein, such as an internal jugular vein (IJV) and particular nerve, such as a vagus nerve and/or median nerve. In some examples, lead 112 may be configured to be positioned percutaneously with respect to an IJV 102 and particular nerve, such as a vagus nerve, median nerve or radial nerve.

In some examples, an ECAP signal is synchronous firing of a population of neurons which occurs in response to the application of a stimulus including, in some cases, an electrical stimulus by a medical device. In some examples, the ECAP signal may be detectable as being a separate event from the stimulus itself, and the ECAP signal may reveal characteristics of the effect of the stimulus on the nerve tissue. An ECAP signal may refer to a measure of the nerve tissue's response to stimulation, such as nerve tissue stimulation therapy. In some examples, ECAPs may be a measure of neural recruitment because each ECAP signal represents the superposition of electrical potentials generated from a population of axons firing in response to an electrical stimulus (e.g., nerve tissue stimulation therapy). Differences in a characteristic (e.g., an amplitude of a portion of the signal or area under the curve of the signal) of ECAP signals may occur as a function of how many axons have been activated by the delivered nerve tissue stimulation therapy.

In some examples, ECAP data may indicate characteristics of activation of particular neural fibers in the nerve tissue. For example, ECAP data may indicate whether B-fibers, which may be targeted to be activated for treatment of heart disease conditions, were activated or C-fibers, which may be targeted to be activated for treatment of epilepsy, were activated. In some examples, IMD 28 and/or computing device 12 may determine what type of neural fibers in the nerve tissue are activated based on the sensed activation signals to determine whether target nerve tissue is sufficiently activated.

For example, in response to a stimulation, such as nerve tissue stimulation therapy, nerve tissue generates an ECAP signal, and the parameters of the ECAP signal, such as an amplitude value, may be a function of how much the nerve tissue responded to particular stimulation, such as the nerve tissue stimulation therapy. In some examples, each electrical stimulation signal in nerve tissue stimulation therapy may elicit an ECAP signal that is sensed by one or more electrodes of a lead 112 that is electrically coupled to IMD 28. In some examples, IMD 28 and/or computing device 12 may receive, via an electrical signal sensed by electrodes of one or more leads 112 information indicative of an ECAP signal (e.g., a numerical value indicating a characteristic of the ECAP signal in electrical units such as voltage or power) produced in response to the nerve tissue stimulation therapy.

In some examples, computing device 12 may determine a nerve tissue stimulation command based on at least one of the EEG signal(s) and/or ECAP(s). In some examples, computing device 12 may determine a nerve tissue stimulation command based on both EEG signal(s) and ECAP(s). In some examples, ECAPS may be referred to as activation signals. For example, IMD 28 and/or computing device 12 may determine that an activation of the nerve tissue satisfies a nerve tissue activation threshold based on the one or more sensed ECAPs satisfying an ECAP threshold. In response to computing device determining that the ECAP signal satisfies an ECAP threshold, IMD 28 may generate an indication that the nerve tissue stimulation therapy by a particular lead 112 is acceptable. For example, in responses to delivery of the nerve tissue stimulation therapy at a particular location, one or more electrodes of a lead 112 may sense an ECAP and IMD 28 and/or computing device 12 may compare the sensed ECAP to an ECAP activation threshold. In response to the sensed ECAP satisfying the ECAP activation threshold, IMD 28 and/or computing device 12 may generate an indication that the nerve tissue stimulation therapy by a particular lead 112 is acceptable. In some examples, in response to the sensed ECAP not satisfying the ECAP activation threshold, IMD 28 may adjust nerve tissue stimulation therapy to be delivered, such as increasing an amount of stimulation to be provided and/or changing a location of a lead 112.

Neurostimulation system 2 may include sensor device 16 that includes or more sensors. In some examples, one or more sensors of sensor device 16 may be one or more of electroencephalography (EEG) sensors, e.g., a plurality of electrodes. Sensor device 16 may additionally or alternatively include one or more sensors to sense an evoked potential (EP) signal, local field potential (LFP) signal, and/or electrocorticography (ECoG) signal of the patient.

In some examples, sensor device 16 may sense EEG signals of patient 14 and transmit the sensed EEG signals to computing device 12. Computing device 12 may receive EEG signals, such as during a first period of time, and determine an initial indication of whether the EEG signals during the first period of time indicate a cranial acute health event, such as stroke, brain ischemia, brain hemorrhage, brain hematoma, brain pressure, and/or hypoxia events.

In some examples, EEG signals fall in the range of 0.5-approximately 200 Hertz (Hz). Waveforms may be subdivided into bandwidths known as delta (δ), theta (θ), alpha (α), beta (β), and gamma (γ). For example, a delta (δ) band may be between 0.5 Hz and 4 Hz, a theta (θ) band may be between 4 Hz and 7 Hz, an alpha (α) band may be between 8 Hz and 12 Hz, a beta (β) band may be between 13 Hz and 30 Hz, and a gamma (γ) band may be between 30 Hz to 200 Hz. In some examples, computing device may determine an initial indication of a cranial acute health event, such as a stroke, based on one or more bandwidth characteristic of the EEG signals, such as an amplitude of a particular bandwidth or a ratio of the energies in two of these bands as a metric, e.g., to determine a change of the value of the amplitude or ratio (or other metric) over a period of time. In some examples, computing device 12 may an initial indication of a cranial acute health event based on particular bandwidth ratios of the EEG signal, such as a delta-alpha ratio (DAR), delta-theta ratio (DTR), a (delta+theta)/(alpha+beta) ratio (DTABR), a beta-alpha ratio (BAR), a gamma-alpha ratio (GAR), and/or a burst-suppression ratio (BSR). In some examples, the respective ratios may be signal power ratios between the respective frequency bandwidths. In some examples, a BSR may be a fraction of an EEG signal spent in a suppressed state (e.g., an amplitude of EEG signal being below a suppressed state threshold, such as less than 5 micro volts) over a period of time.

In some examples, computing device 12 may transmit a nerve tissue stimulation command to IMD 28 in response to a determination that the EEG signals during the first period of time indicate a cranial acute health event. In some examples, a nerve tissue stimulation command may include one or more of protocols, parameters, or regimes of electrical signal to be applied by IMD 28 to stimulate nerve tissue. IMD 28 may receive the nerve tissue stimulation command and apply an electrical signal to stimulate nerve tissue based on the received nerve tissue stimulation command. For example, IMD 28 may apply an electrical signal to stimulate a median nerve of patient 14. In some examples, sensor device 16 may sense an EP signal and determine an EP based on the sensed EP signal. In some examples, sensor device 16 may sense an EP signal during a second period of time, the second period of time being after the first period of time and after IMD 28 applies an electrical signal to stimulate nerve tissue, such as after IMD 28 applies an electrical signal to stimulate a median nerve. In some examples, sensor device 16 may transmit the EP to computing device 12. In some examples, computing device 12 may receive an EP, the EP occurring during the second period of time. In some examples, EP may be somatosensory evoked potential (SSEP). In some examples, features of the EP, such as amplitude, may indicate how well a nerve, such as a median nerve, responds to stimulation.

In some examples, computing device 12 may determine/confirm the initial indication of a cranial acute health event is a cranial acute health event based on one or more parameters of the EP satisfying a cranial acute health event EP threshold. For example, a cranial acute health event EP threshold may be an amplitude of EP that is 50% of a pre-determined healthy EP amplitude level and the cranial acute health event EP threshold is satisfied if an amplitude of the determined EP is less than 50% of the pre-determined healthy amplitude level. In some examples, computing device 12 may determine a personalized cranial acute health event threshold based on sensing an initial EP signal during a period of time that is before the initial indication of a cranial acute health event and that are also in response to electrical signals being applied by IMD 28 to stimulate nerve tissue, such as a median nerve, during the period of time that is before the initial indication of a cranial acute health event.

In some examples, in response to a determination by computing device 12 that the initial indication of a cranial acute health event is a cranial acute health event, computing device 12 may transmit a second nerve tissue stimulation command to IMD 28 to apply an electrical signal to stimulate nerve tissue, such as vagus nerve tissue.

In some examples, using the techniques as described above, neurostimulation system 2 may determine, via EEG signals sensed by sensor device 16, an initial indication of a cranial acute health event, stimulate, via IMD 28, a median nerve of patient 14, determine, via EP signals sensed by sensor device 16, that one or more parameters of an EP, such as SSEP, satisfy a cranial acute health event threshold, and stimulate, via IMD 28, a vagus nerve of patient 14 in response to a determination that the one or more parameters of an EP satisfy a cranial acute health event threshold. Thus, neurostimulation system 2 is able to timely apply treatment, such as electrical stimulation to vagus nerve tissue, in instances in which the initial indication of a cranial acute health event is detected and then confirmed, which allows the neurostimulation system 2 to provide improved treatment to patients suffering from cranial acute health event while also reducing instances in which vagus nerve tissue stimulation is provided in response to a false positive detection of a cranial acute health event.

In addition, neurostimulation system 2 may provide an improved nerve tissue stimulation treatment based on determining a nerve tissue stimulation command based on sensed EEG signals and sensed ECAPs. For example, by applying electrical signals to stimulate nerve tissue based on sensed EEG signals and sensed ECAPs, neurostimulation system 2 may stimulate particular nerve tissue, such as the vagus nerve, more precisely to generate improved therapeutic results.

Computing device 12 may be configured for wireless communication with IMD 28 and/or sensor device 16. In addition, IMD 28 may be configured for wireless communication with sensor device 16. Computing device 12 may retrieve data from IMD 28 that was collected and stored by the IMD 28. In some examples, computing devices 12 may take the form of personal computing devices of patient 14. For example, computing device 12 may take the form of a smartphone of patient 14, and/or a smartwatch or other smart apparel of patient 14. In some examples, computing device 12 may be any computing device configured for wireless communication with IMD 28 such as a desktop, laptop, or tablet computer. In some examples, computing device may be a dedicated programming device for IMD 28, e.g., a clinician programmer or a patient programmer. Computing device 12 may communicate with IMD 28 according to standards or protocols, such as 3G, 4G, 5G, WiFi (e.g., 802.11 or 802.15 ZigBee), Bluetooth®, or Bluetooth® Low Energy (BLE).

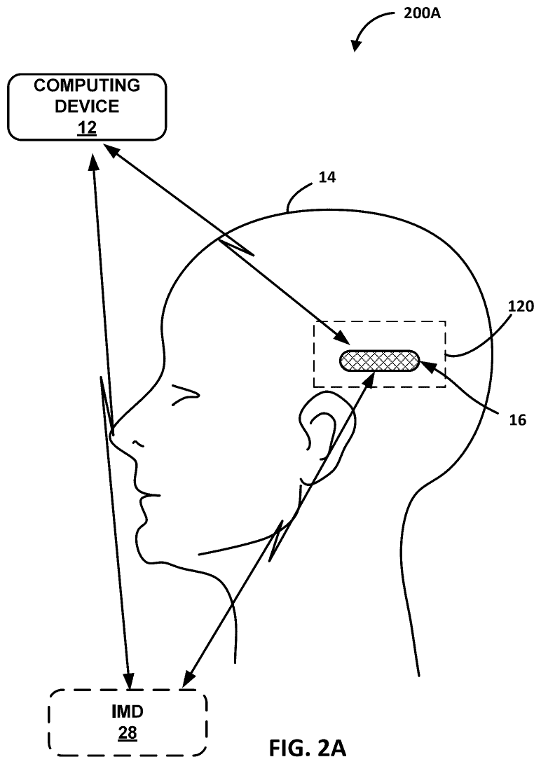

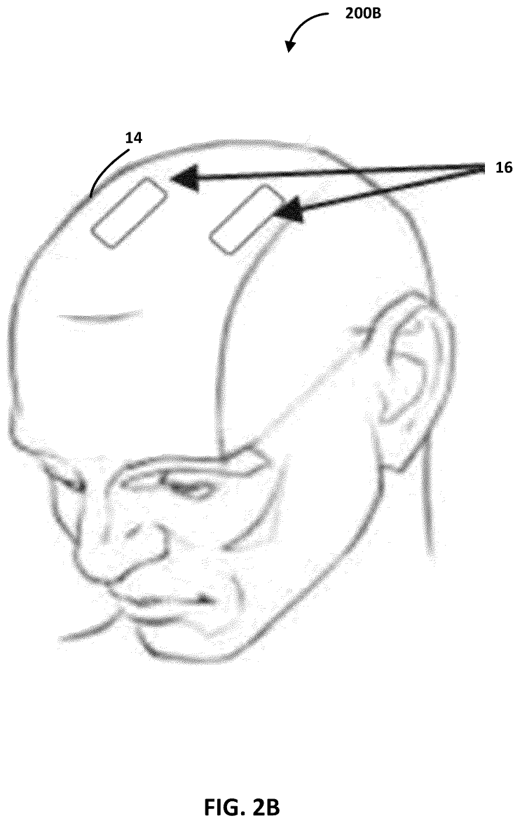

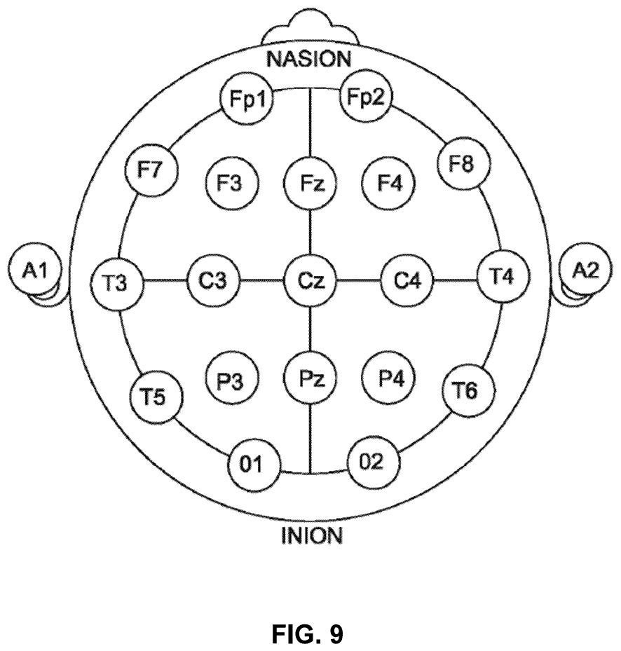

FIG. 2A a conceptual diagram of a system 200A configured to determine an indication of a cranial acute health event in accordance with examples of the present disclosure. System 200A may be substantially similar to system 2 of FIG. 1. However, sensor device 16 may be configured to be implanted in target region 120 which is located on the side of the head posterior of the temple of patient 14, e.g., above the ear and/or over the temporal portion of the cranium. Sensor device 16 implanted at target region 120 may be configured to generate cranial acute health event metrics based on electrical signals sensed in this area. In such examples, the electrodes of sensor device 16 may detect electrical activity that corresponds to brain activity in the T3 region (as shown in FIG. 9), or T4 region if implanted on the other side of the patient's head, or both of two or more sensor devices are implanted bilaterally at temporal regions. In some examples, sensor device 16 may employ different filters or other processing or signal conditioning techniques than those at a different target region due to different types of noise at target region 120, such as muscle activity due to mandible movement or other types of electrical activity. In some examples, additionally or alternatively, computing device 12 may employ different filters or other processing or signal conditioning techniques than those at a different target region due to different types of noise at target region 120, such as muscle activity due to mandible movement or other types of electrical activity.

FIG. 2B is a conceptual diagram of a system 200B configured to determine an indication of a cranial acute health event in accordance with examples of the present disclosure. System 200B may be substantially similar to system 2 of FIG. 1 or system 200A of FIG. 2A. However, system 200B may be configured to include a plurality of IMDs 16, such as two or more sensor devices 16, to be located on the head of patient 14.

Each of sensor devices 16 may include a respective set of electrodes and be configured to sense respective EEG signals and/or EP signals via the respective electrode (and/or other physiological parameters via other sensors or the electrodes as described herein). In the example, illustrated in FIG. 2B, sensor devices 16 (and consequently their respective electrodes) are positioned to detect EEG signals of respective areas, e.g., hemispheres, of the brain of patient 14. Systems (e.g., processing circuitry) described herein may use different localized EEG signals and/or EP signals to localize the cranial acute health event, such as stroke, e.g., to a particular hemisphere, or for other purposes related to diagnosing such events as described herein. In some examples, a single sensor device may include electrodes coupled thereto via extensions, which may be positioned in different hemispheres or other regions to similar acquire localized EEG signals.

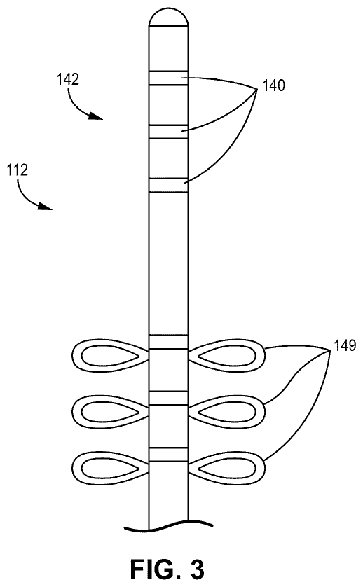

FIG. 3 is an example of a conceptual diagram of a lead 112. Lead 112 may include a plurality of electrodes 140 and a plurality of anchoring mechanisms 149. Electrodes 140 may be referred to as stimulating electrodes, sensing electrodes, or stimulating/sensing electrodes. In some examples, electrodes 140 may be configured to deliver stimulation signal(s) and/or sense electrical activity, such as ECAPs that may represent the firing of a population of electrically stimulated nerve fibers of a particular nerve. In some examples, electrodes 140 may be segmented electrodes. In some examples, electrodes 140 may be current steerable segmented electrodes. For example, lead 112 may include current steerable segmented electrodes that may steer current directly at a particular nerve, such as a particular vagus nerve or median nerve, which may help minimize collateral nerve capture. Anchoring mechanisms 149 may be configured to provide endovascular anchoring, such as in an internal jugular vein (IJV). For example, lead 112 may include anchoring mechanisms to hold lead 112 in an IJV next to a particular nerve to be stimulated and/or sensed, such as a particular vagus nerve or median nerve. In an example, anchoring mechanism(s) 149 may safely secure, or provide stability for, a portion of a respective lead 112 to a wall of a blood vessel inside the body of the patient. By securing or increasing stability, anchoring mechanism(s) 149 may prevent unwanted movement of the respective lead 112 out of a targeted location in the body.

In some examples, lead 112 may be self-expanding stent like or braided structures with one or more electrodes arranged on the outside surface of the structure, such that the electrode is placed against the inside wall of respective IJV when the structure expands. In some examples, lead 112 may be one or more ring like structures with one or more electrodes arranged on the outside surface of the structure, or with the entire surface of one or more rings serving as an electrode, such that the electrodes are placed against the inside wall of the respective IJV when the structure expands. In some examples, lead 112 may be a loop structure (e.g. similar to an Achieve™ mapping catheter from Medtronic, Inc.) with one or more electrodes arranged on the outside surface of the structure, such that the electrode is placed against the inside wall of the respective IJV when the structure expands. In some examples, lead 112 may be a helix structure with one or more electrodes arranged on the outside surface of the structure, such that the electrode is placed against the inside wall of the respective IJV when the structure expands.

Electrodes 140 are positioned at distal end or portion 142 of lead 112. Anchoring mechanisms 149 may also be, but are not necessarily, positioned at the distal end or portion 142, e.g., proximate to electrodes 140, to anchor the position/orientation of electrodes 140 relative to a particular nerve, such as a particular vagus nerve or media nerve. In the illustrated example, anchoring mechanisms 149 are positioned proximally on lead 112 with respect to the position of electrodes 140 at distal end or portion 142 of lead 112. As an example, lead 112 being configured as described above may provide greater maneuverability and allow temporary trialing patients to test therapy responsiveness. In some examples, anchoring mechanisms 149 may be lantern-shaped, tines, and/or other shapes.

The anchoring mechanisms may include a passive anchoring mechanism, an active anchoring mechanism or a combination of both. In one example, the anchoring mechanism is coupled at a distal end of the lead body and may also function as an electrically active element. Examples of passive anchoring mechanisms include flanges, flared ends of expandable stents or braids, hoops/rings/helices providing wall apposition within the IJV, disks, pliant tines, flaps, bio-adhesive surfaces, and/or any other non-piercing elements. Examples of active anchoring mechanisms may include rigid tines, prongs, barbs, hooks, clips, screws, and/or other projecting elements that pierce and penetrate into tissue to anchor the lead. As another example of an active anchoring mechanism, the lead may be provided with a side helix for engaging tissue.

The various examples of the anchoring mechanisms may be deployable. As such, the anchoring mechanism assumes a first state during maneuvering of the lead (during which time the lead is disposed within a lumen of a delivery system or overtop a guidewire or stylet) to the desired implant location. Subsequently, the anchoring mechanism assumes a second state following the release of the lead from the delivery system into the substernal space to thereby anchor the distal end portion of the lead body relative to the adjacent tissue. Electrodes may be positioned anywhere with respect to the anchoring mechanisms such that they are held against the inner wall of the IJV by the anchoring system, and may be an integral part of the anchoring system.

Neurostimulation system 2 may deliver stimulation signal(s) to patient 14 by IMD 28 generating and delivering a programmable electrical stimulation signal (e.g., in the form of electrical pulses or an electrical waveform) to a therapy site near where electrodes 140 are disposed. The distal end of a respective lead 112 may be inserted into patient 14 in such a manner as to locate electrodes 140 in a respective IJV near a respective nerve of patient 14. Lead 112 may be constructed of biocompatible materials.

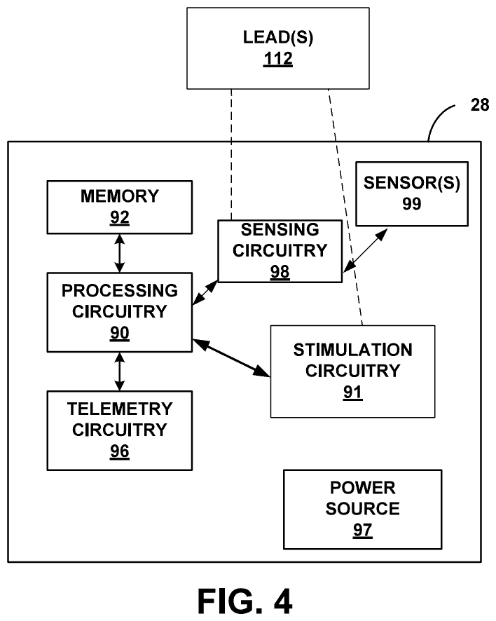

Lead 112 may include a plurality of electrodes 140. It is understood that any appropriate number of electrodes 140n may be provided. Each of the electrodes 140 may be electrically coupled to the stimulation circuitry 98 (FIG. 4) and may be controlled individually and/or in combination to provide stimulation signal(s) to one or more portions of a respective nerve. The stimulation signal(s) through the electrodes 140 may be provided in any appropriate manner, such as discussed further herein. In various examples, for example, one of the electrodes 140, may be operated as an anode while a second electrode may be operated as a cathode. Thus, a voltage differential may be generated between the two electrodes to provide stimulation to one or more portions of the particular nerve to be stimulated, such as a particular vagus nerve or median nerve.

In some examples, two or more of electrodes 140 may be used to sense an electrical signal indicative of an ECAP signal (e.g., a numerical value indicating a characteristic of the ECAP signal in electrical units such as voltage or power) produced in response to nerve tissue of a particular nerve being stimulated, such as by lead 112.

FIG. 4 is a block diagram illustrating an example configuration of IMD 28. As illustrated in FIG. 4, IMD 28 may include one or more of processing circuitry 90, stimulation circuitry 91, memory 92, telemetry circuitry 96, power source 97, sensing circuitry 98, or sensor(s) 99. In some examples, a combination of one or more of processing circuitry 90, stimulation circuitry 91, telemetry circuitry 96, and sensing circuitry 98 in IMD 28 may generally be referred to as circuitry. Memory 92 may store program instructions that, when executed by processing circuitry 90, cause processing circuitry 90 to provide the functionality ascribed to IMD 28 throughout this disclosure. In general, IMD 28 may include any suitable arrangement of hardware, alone or in combination with software and/or firmware, to perform the techniques attributed to IMD 28, and processing circuitry 90, stimulation circuitry, and telemetry circuitry 96 of IMD 28. In various examples, IMD 28 may include one or more processors, such as one or more microprocessors, DSPs, ASICs, FPGAs, or any other equivalent integrated or discrete logic circuitry, as well as any combinations of such components. IMD 28 also, in various examples, may include a memory 92, such as RAM, ROM, PROM, EPROM, EEPROM, flash memory, a hard disk, a CD-ROM, comprising executable instructions for causing the one or more processors to perform the actions attributed to them. Moreover, although processing circuitry 90 and telemetry circuitry 96 are described as separate circuitry, in some examples, processing circuitry 90 and telemetry circuitry 96 are functionally integrated. In some examples, processing circuitry 90 and telemetry circuitry 96 correspond to individual hardware units, such as microprocessors, ASICs, DSPs, FPGAs, or other hardware units. In other examples, any of processing circuitry 90 and telemetry circuitry 96 may correspond to multiple individual hardware units, such as microprocessors, ASICs, DSPs, FPGAs, or other hardware units.

In some examples, memory 92 may further include program information, e.g., stimulation programs defining the neurostimulation. Generally, stimulation circuitry 91 may generate and deliver electrical stimulation under the control of processing circuitry 90. In some examples, processing circuitry 90 controls stimulation circuitry 91 by accessing memory 92 to selectively access and load at least one of the stimulation programs to stimulation circuitry 91. For example, in operation, processing circuitry 90 may access memory 92 to load a stimulation program to stimulation circuitry 91. In other examples, stimulation circuitry 91 may access memory 92 and load one of the stimulation programs. In some examples, the electrical stimulation signal(s) generated and delivered by stimulation circuitry 91 may be around 10 Hz.

In some examples, stimulation programs may include stimulation programs that are configured to facilitate different effects. For example, stimulation circuitry may use different stimulation programs to generate different electrical stimulation signals to cause different effects. In some examples, stimulation circuitry 91 may generate an electrical stimulation signal in the range of about 1 to 100 Hz or generate an electrical stimulation signal in the range of about 500 Hz to about 50 kHz. Stimulation circuitry 91 may deliver stimulation signal(s) to patient 14 for an extended period of time, such as minutes, hours, days, or until patient 14 or a clinician manually stops or changes the stimulation.

Stimulation circuitry 91 may deliver stimulation signal(s) according to stimulation parameters. Stimulation circuitry 91 may be electrically coupled to one or more leads 112. In some examples, stimulation circuitry 91 may deliver stimulation signal(s) in the form of electrical pulses. In such examples, relevant stimulation parameters may include a voltage amplitude, a current amplitude, a pulse rate, a pulse width, a duty cycle, a duty cycle of the stimulation ON/OFF periods, or the combination of electrodes 140 and/or leads 112 that stimulation circuitry 91 uses to deliver stimulation signal(s). In other examples, stimulation circuitry 91 may deliver stimulation signal(s) in the form of continuous waveforms. In such examples, relevant stimulation parameters may include a voltage or current amplitude, a frequency, a shape of the stimulation signal, a duty cycle of the stimulation signal, or the combination of electrodes 140 and/or leads 112 stimulation circuitry 91 uses to deliver the stimulation signal(s).

Sensing circuitry 98 may be electrically coupled to one or more leads 112. Sensing circuitry 98 may be electrically coupled to some or all of electrodes 140. Sensing circuitry 98 may be coupled to some or all of sensor(s) 99. While FIG. 4 shows sensor(s) 99 as a part of IMD 28, in some examples, one or more sensor(s) 99 may be positioned external to IMD 28 but are communicatively coupled to IMD 28 via the telemetry circuitry 96. Sensing circuitry 98 is configured to obtain signals, such as electrical signals indicative of ECAPS, sensed via one or more combinations of electrodes 140 and/or sensor(s) 99 and process the obtained signals. Sensing circuitry 98 may include one or more filters. In some examples, sensing circuitry 98 may be implemented in the processing circuitry 90 of IMD 28. The components of sensing circuitry 98 may be analog components, digital components or a combination thereof. Sensing circuitry 98 may, for example, include one or more sense amplifiers, filters, rectifiers, threshold detectors, analog-to-digital converters (ADCs) or the like. Sensing circuitry 98 may convert the sensed signals to digital form and provide the digital signals to processing circuitry 90 for processing or analysis. For example, sensing circuitry 98 may amplify signals from the sensing electrodes and convert the amplified signals to multi-bit digital signals by an ADC.

Telemetry circuitry 96 supports wireless or wired communication between IMD 28, computing device 12, sensor device 16, 110, 210, and/or any other device, such as an implantable cardiac monitor, under the control of processing circuitry 90. Telemetry circuitry 96 may also be configured to communicate with another computing device via wireless communication techniques, or direct communication through a wired connection. In some examples, telemetry circuitry 96 may provide wireless communication via an RF, proximal inductive medium, or Tissue Conductance Communication (TCC). In some examples, telemetry circuitry 96 may include an antenna, which may take on a variety of forms, such as an internal or external antenna. In some examples, telemetry circuitry 96 may provide communication according to standards or protocols, such as 3G, 4G, 5G, WiFi (e.g., 802.11 or 802.15 ZigBee), Bluetooth®, or Bluetooth® Low Energy (BLE). In some examples, telemetry circuitry 96 may provide communication through relay, such as through a proprietary telemetry protocol relayed by a standard protocol, such as BLE.

Examples of local wireless communication techniques that may be employed to facilitate communication between IMD 28 and another computing device include RF communication according to the 802.11 or Bluetooth specification sets, infrared communication, e.g., according to the IrDA standard, or other standard or proprietary telemetry protocols. In this manner, other external devices may be capable of communicating with IMD 28 without needing to establish a secure wireless connection.

Power source 97 delivers operating power to the components of IMD 28. Power source 97 may include a battery and a power generation circuit to produce the operating power. In some examples, the battery may be rechargeable to allow extended operation.

In some examples, circuitry of IMD 28, e.g., stimulation circuitry 91, may be configured to deliver the electrical energy to at least one lead 112 to deliver the respective stimulation signal(s) in response to receiving a nerve tissue stimulation command, such as a nerve tissue stimulation command determined based on at least one of the EEG signals and the ECAPs, a nerve tissue stimulation command based on determining an initial indication of a cranial acute health event based on EEG signals, and/or a nerve tissue stimulation command based on determining/confirming the initial indication of the cranial acute health event is a cranial acute health event. In some examples, circuitry of IMD 28 may be configured to adjust the delivery of electrical energy, such as a level of electrical energy being, a duration of delivery of electrical, or other variables of electrical energy delivery that may be adjust, to at least one lead 112 to deliver the stimulation signal(s) based on features of the EEG signals and/or ECAPS, one or more parameters of an initial indication of a cranial acute health event, and/or one or more parameters of determination/confirmation the initial indication of the cranial acute health event is a cranial acute health event, such as parameters of the EP. In some examples, neuromodulation system 2 may provide a closed-loop system in determining/confirming a cranial acute health event and providing/adjusting vagus nerve tissue stimulation based on parameters of the cranial acute health event, which may lead to improved patient health.

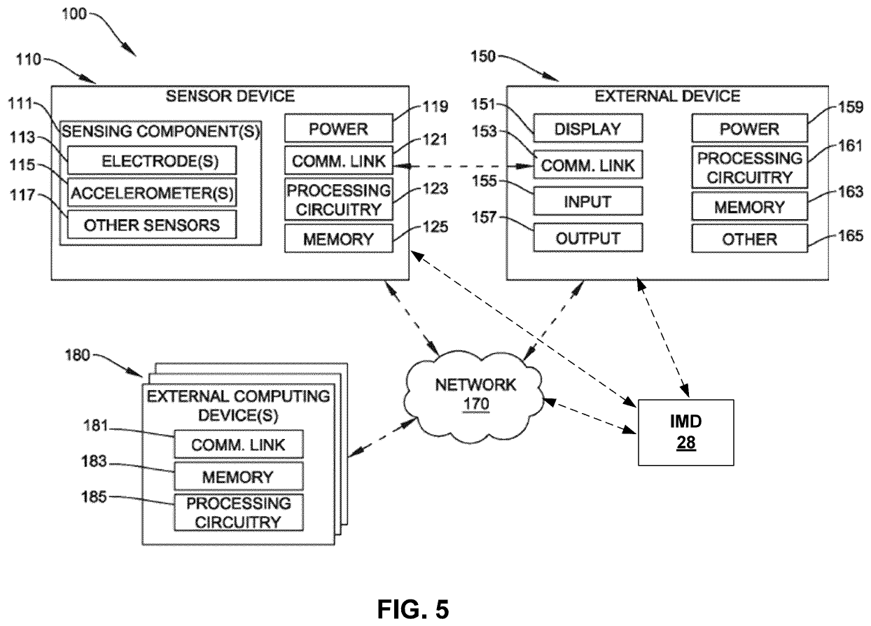

In some examples, neurostimulation system 2 may further include a sensor device 16 communicatively coupled to IMD 28. In some examples, neurostimulation system 2 may further include a sensor system including one or more sensor devices 16 communicatively coupled to IMD 28. In some examples, sensor device 110, 210, 310 may include some or all of the features of the sensor device 16 describe above with respect to FIG. 1. Some examples of sensor device 110, 210, 310 are further described below with respect to FIGS. 5-9.

FIG. 5 is a schematic diagram of an example sensor system 100. Although the sensor system 100 is shown with certain devices for purposes of explanation, in various examples any one or more of the devices shown in FIG. 5 can be omitted. Similarly, although the devices shown in FIG. 5 are illustrated as including certain components, in various examples any one or more of the particular components within these devices can be omitted (e.g., the sensor device 110 may omit the accelerometer 115). Moreover, any of the devices can include additional components not specifically shown here. In some examples, sensor system 100 may include sensor device 110, IMD 28, external device 150 and/or external computing device(s) 180 communicatively coupled to at least one of each other.

The sensor system 100 can be configured to sense physiological patient data and analyze that data to make a cranial acute health event determination. In an example, the sensor system 100 includes a sensor device 110 that is configured to be implanted in a target site of the patient or disposed over the skin of the patient at a target site. In some examples, the sensor device may be a relatively small device, and may be placed (e.g., inserted) under or over the skin at the back of the patient's neck or base of the skull. As described in more detail below, the sensor device 110 may detect one more physiological parameters of a patient (e.g., electrical activity corresponding to brain activity in particular regions of the patient's brain, ECG data, motion data, etc.). The sensor device 110 may be communicatively coupled to an external device 150, for example via a wireless connection. In some examples, the external device 150 can be a mobile device (e.g., a smartphone, tablet, smartwatch, etc.) or other computing device with which the patient can interact. In operation, the patient may receive output or instructions from the external device 150 that are based at least in part on data received at the external device 150 from the sensor device 110. For example, the external device 150 may provide an alert to the patient or another entity (e.g., a call center) based on a stroke indication provided by the sensor device 110. Additionally or alternatively, the external device 150 may output user prompts which can be synchronized with data collection via the sensor device 110. For example, the external device 150 may instruct the user to lift an arm, make a facial expression, etc., and the sensor device 110 may record physiological data while the user performs the requested actions. Moreover, the external device 150 may itself analyze the patient (e.g., the patient's activity or condition in response to such prompts), for example using a camera to detect facial drooping, using a microphone to detect slurred speech, or to detect any other indicia of a cranial acute health event. In some examples, such indicia can be compared against pre-cranial acute health event inputs (e.g., a stored baseline facial image or voice-print with baseline speech recording).

The sensor device 110 may also be communicatively coupled with IMD 28. IMD 28 may receive information from sensor device 110 via external device 150. For example, IMD 28 may communicate directly with external device 150 which communicates with sensor device 110. In some examples, IMD 28 may communicate directly with sensor device 110 and receive information directly from sensor device 110. In some examples, sensor device 110, external device 150, and/or IMD 28 may be communicatively coupled with each other over network 170.

The sensor device 110 and/or the external device 150 can also be communicatively coupled with one or more external computing devices 180 (e.g., over network 170). In some examples, the external computing devices 180 can take the form of servers, personal computers, tablet computers or other computing devices associated with one or more healthcare providers (e.g., hospitals, medical data analytic companies, device manufacturers, etc.). These external computing devices 180 can collect data recorded by the sensor device 110 and/or the external device 150. In some examples, such data can be anonymized and aggregated to perform large-scale analysis (e.g., using machine-learning techniques or other suitable data analysis techniques) to develop and improve stroke detection algorithms using data collected by a large number of sensor devices 110. Additionally, the external computing devices 180 may transmit data to the external device 150 and/or the sensor device 110. For example, an updated algorithm for making stroke determinations may be developed by the external computing devices 180 (e.g., using machine learning or other techniques) and then provided to the sensor device 110 and/or the external device 150 via the network (e.g., as an over-the-air update), and installed on the sensor device 110 and/or external device 150.

In some examples, the sensor system 100 may also include additional implantable devices, such as IMD 28, implantable cardiac monitors, an implantable pacemaker, an implantable cardiac defibrillator, a cardiac resynchronization therapy (CRT) device (e.g., CRT-D defibrillator or CRT-P pacemaker), a neurostimulator, a deep-brain stimulation device, a nerve stimulator, a drug pump (e.g., an insulin pump), a glucose monitor, or other devices. Other devices that may support and enhance a personal ecosystem to reduce stroke risk include fitness monitors, nutrition devices, etc. Additionally or alternatively, a cranial acute health event detection device can be used in conjunction with other disease therapies with high risk of cranial acute health event as an adverse event (e.g., LVAD devices, TAVI/TAMR devices, bariatric/gastric surgery, etc.). In some examples of this disclosure, a system including both sensor device 110 and IMD 28 may be referred to as neurostimulation system 2 and/or sensor system 100.

As noted previously, the sensor device 110 is configured to be coupled to a patient for recording physiological data relevant to a cranial acute health event determination. For example, the sensor device 110 can be implanted within the body of a patient, may be disposed directly over a patient's skin (e.g., held in place via an adhesive or fastener), or may be removably worn by the patient. The sensor device 110 includes sensing components 111, which can include a number of different sensors and/or types of sensors. For example, the sensing components 111 can include a plurality of electrodes 113, an accelerometer 115, and optionally other sensors 117. Examples of other sensors 117 include an EP sensor, a blood pressure sensor, a pulse oximeter, an ECG sensor or other heart-recording device, an EMG sensor or other muscle-activity recording device, a temperature sensor, a skin galvanometer, hygrometer, altimeter, gyroscope, magnetometer, proximity sensor, hall effect sensors, or any other suitable sensor for monitoring physiological characteristics of the patient. These particular sensing components 111 are exemplary, and in various examples the sensors employed can vary.

The electrodes 113 may be configured to detect electrical activity such as brain activity (e.g., EEG data), EP signals, heart activity (e.g., ECG data), and/or muscle activity (e.g., EMG data). The electrodes 113 may be formed from any suitable conductive material or materials to enable the electrodes to perform electrical measurements on the patient.

In some examples, the sensor device 110 is configured to analyze data from the electrodes 113 to extract brain activity data and/or EP data and to discard or reduce any contribution from heart or muscle activity. In some examples, the electrodes 113 are configured to be disposed over the patient's skin. In such examples, the electrodes 113 can include protrusions (e.g., microneedles or other suitable structures) configured to at least partially penetrate the patient's skin so as to improve detection of subcutaneous electrical activity. In some examples, the sensor device 110 can be configured to be implanted within the body (e.g., subcutaneously), and as such the electrodes 113 can include a conductive surface exposed along at least a portion of the sensor device 110 so as to detect electrical activity within the body.

The sensor device 110 may be configured to calculate physiological characteristics relating to one or more electrical signals received from the electrodes 113. For example, the sensor device 110 may be configured to algorithmically determine the presence or absence of a cranial acute health event, such as a stroke, or other neurological condition from the electrical signal. In some examples, the sensor device 110 may make a cranial acute health event determination for each electrode 113 (e.g., channel) or may make a cranial acute health event determination using electrical signals acquired from two or more selected electrodes 113.

In various examples, the number and configuration of electrodes 113 can vary. For example, the sensor device 110 may include at least 2, at least 3, at least 4, at least 5, or more electrodes 113 in an array. In some examples, the sensor device 110 includes fewer than 6, fewer than 5, fewer than 4, or fewer than 3 electrodes 113 in an array. As described in more detail below, although conventional EEG arrays include a large number of electrodes disposed over the top of a patient's head, some examples include a relatively small number of electrodes (e.g., three electrodes) configured to be placed over the back of the patient's neck or base of the skull. In this position, electrical data collected via these electrodes 113 may correspond to brain activity in regions determined to be of interest for stroke determination (e.g., the P3, Pz, and/or P4 regions).

In some examples, the electrodes 113 may all reside within a single housing of the sensor device 110. In some examples, the electrodes 113 may extend away from a housing of the sensor device 110 and be connected via leads or other connective components. For example, the sensor device 110 can include a housing that encompasses certain components (e.g., the power source 119, communications link 121, processing circuitry 123, and/or memory 125), and the electrodes 113 (and/or other sensing components 111) can be coupled to the housing via electrical leads or other suitable connections. In such configurations, the electrodes 113 can be positioned at locations spaced apart from the housing of the sensor device 110. In some examples, the electrodes 113 can be disposed within discrete housings that are in turn coupled to a housing containing the other components of the sensor device 110. Such a configuration, in which multiple housings (or sub-housings) are coupled together via flexible or other connectors, may facilitate placement of the sensor device 110 at a desired location to improve patient comfort. Additionally, this may facilitate placement of electrodes 113 at desirable positions for detecting clinically useful brain activity data.

The accelerometer 115 can be configured to detect patient movement. In some examples, patient movement data collected via the accelerometer 115 can be used to make a fall determination.

The sensor device 110 can also include a power source 119 (e.g., a battery, capacitors). In some examples, the power source 119 can be rechargeable, for example using inductive charging or other wireless charging techniques. Such rechargeability can facilitate long-term placement of the sensor device 110 on or within a patient.

A communications link 121 enables the sensor device 110 to transmit to and/or receive data from external devices (e.g., external device 150 or external computing devices 180). The communications link 121 can include a wired communication link and/or a wireless communication link (e.g., Bluetooth, Near-Field Communications, LTE, 5G, Wi-Fi, infrared and/or another wireless radio transmission network).

The processing circuitry 123 can include one or more CPUs, ASICs, digital signal processing circuitry, or any other suitable electrical components configured to process data from the sensing components 111 and control operation of the sensor device 110. In some examples, the processing circuitry 123 includes hardware particularly adapted for artificially intelligence and/or machine learning applications, for example, a tensor processing unit (TPU) or other such hardware. In certain examples, the processing circuitry of the sensor device 110 may include one or more input protection circuits to filter the electrical signals and may include amplifier/filter circuitry to remove DC and high frequency components, one or more analog-to-digital (A/D) converters, or any other suitable components.

The sensor device 110 can further include memory 125, which can take the form of one or more computer readable storage modules configured to store information (e.g., signal data, subject information or profiles, environmental data, data collected from one or more sensing components, media files) and/or executable instructions that can be executed by the processing circuitry 123. The memory 125 can include, for example, instructions for analyzing patient data to determine whether a patient is undergoing or has recently or previously undergone a cranial acute health event. In some examples, the memory 125 stores data (e.g., signal data acquired from the sensing components 111) used in the cranial acute health event detection techniques disclosed herein.

As noted above, in some examples, the sensor device 110 may also communicate with an external device 150. The external device 150 can be, for example, a smartwatch, smartphone, laptop, tablet, desktop PC, or any other suitable computing device and can include one or more features, applications and/or other elements commonly found in such devices. For example, the external device 150 can include display 151, a communications link 153 (e.g., a wireless transceiver that may include one or more antennas for wirelessly communicating with, for example, other devices, websites, and the sensor device 110). Communication between the external device 150 and other devices can be performed via, e.g., a network 170 (which can include the Internet, public and private intranet, a local or extended Wi-Fi network, cell towers, the plain old telephone system (POTS), etc.), direct wireless communication, etc. The external device 150 can additionally include well-known input components 131 and output components 133, including, for example, a touch screen, a keypad, speakers, a camera, etc.

In operation, the patient may receive output or instructions from the external device 150 that are based at least in part on data received at the external device 150 from the sensor device 110. For example, the sensor device 110 may generate a stroke indication based on analysis of data collected via sensing components 111. The sensor device 110 may then instruct the external device 150 to output an alert to the patient (e.g., via display 151 and/or output 157) or another entity. In some examples, the alert can both be displayed to the user (e.g., via display 151 of the external device) and can also be transmitted to an appropriate emergency medical response service (e.g., a 9-1-1 call may be placed with location data from the external device 150 used to direct responders to locate the patient), and/or to other healthcare provider entities or individuals (e.g. a hospital, emergency room, or physician). In some examples, embedded circuitry that provides location data (e.g., a GPS unit) can be included within the sensor device 110.

Additionally or alternatively, the external device 150 may output user prompts which may be used in conjunction with physiological data collection via the sensor device 110. For example, the external device 150 may instruct the user to perform an action (e.g., lift an arm, make a facial expression, etc.), and the sensor device 150 may record physiological data while the user performs the requested actions. In some examples, the external device 150 may itself analyze physiological parameters of the patient, for example using a camera to detect facial drooping or other indicia of stroke. In some examples, such physiological data collected via the external device 150 can be combined with data collected via the sensing components 111 and analyzed together to make a stroke determination.

As noted previously, the external computing device(s) 180 may take the form of servers or other computing devices associated with healthcare providers or other entities. The external devices can include a communications link 181 (e.g., components to facilitate wired or wireless communication with other devices either directly or via the network 170), a memory 183, and processing circuitry 185. These external computing devices 180 can collect data recorded by the sensor device 110 and/or the external device 150. In some examples, such data can be anonymized and aggregated to perform large-scale analysis (e.g., using machine-learning techniques or other suitable data analysis techniques) to develop and improve cranial acute health event detection algorithms using data collected by a large number of sensor devices 110 associated with a large population of patients. Additionally, the external computing devices 180 may transmit data to the external device 150 and/or the sensor device 110. For example, an updated algorithm for making cranial acute health event determinations may be developed by the external computing devices 180 (e.g., using machine learning or other techniques) and then provided to the sensor device 110 and/or the external device 150 via the network 170, and installed on the recipient device 110/150.

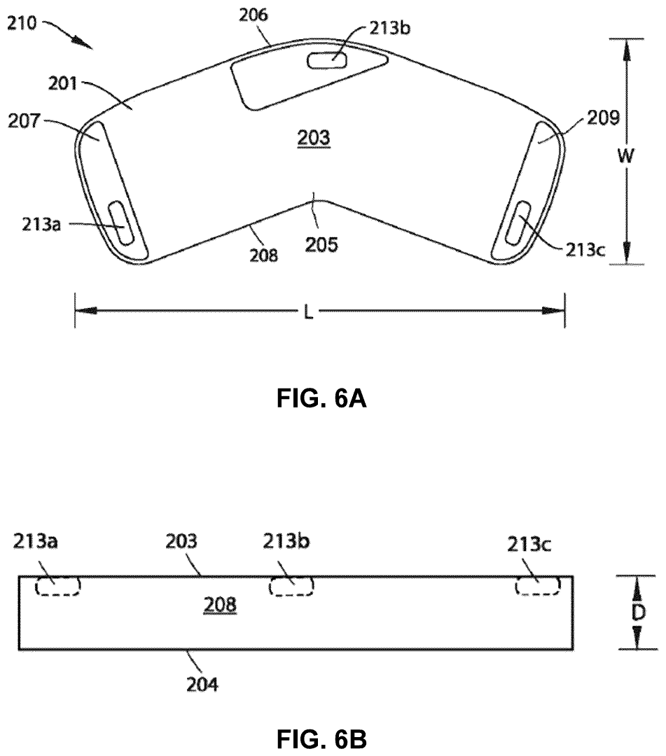

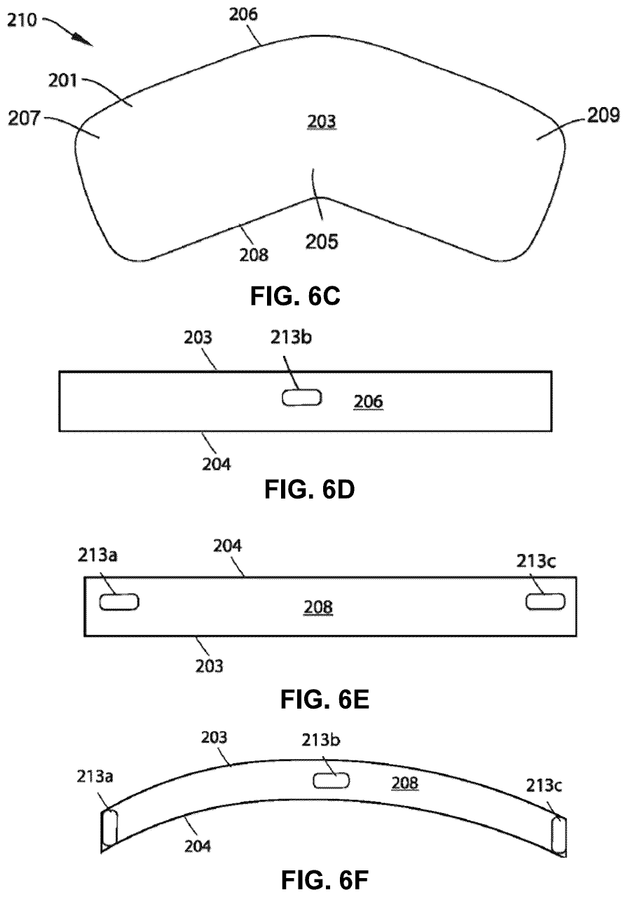

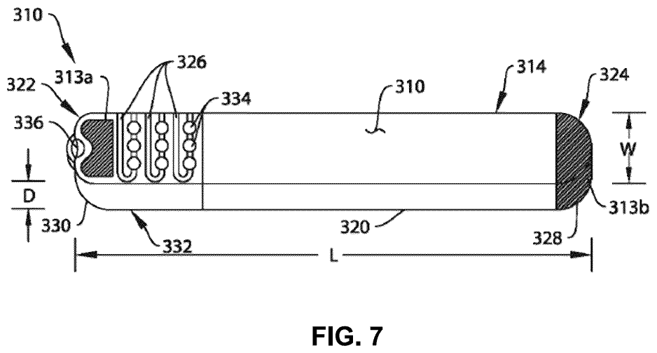

FIG. 6A illustrates a plan view of an example sensor device 210. In some examples, the sensor device 210 can include some or all of the features of the sensor device 16 describe above with respect to FIG. 1, the sensor device 110 described above with respect to FIG. 5 and/or the sensor device 310 described below with respect to FIG. 7, and can include additional features as described in connection with FIG. 6A. In some examples, sensor device 210 is an example of sensor device 16 shown in FIG. 1. In the illustrated example, the sensor device 210 includes a housing 201 that carries a plurality of electrodes 213a-c (collectively “electrodes 213”) therein. In operation, the electrodes 213a-c can be placed in direct contact with tissue at the target site (e.g., with the user's skin if placed over the user's skin, or with subcutaneous tissue if the sensor device 210 is implanted). The housing 201 additionally encloses electronic circuitry located inside the sensor device 210 and protects the circuitry contained therein from body fluids. In various examples, the electrodes 213 can be disposed along any surface of the sensor device 210 (e.g., anterior surface, posterior surface, left lateral surface, right lateral surface, superior side surface, inferior side surface, or otherwise), and the surface in turn may take any suitable form.

In the example of FIGS. 6A and 6B, the housing 201 may be a biocompatible material having a relatively planar shape including a first major surface 203 configured to face towards the tissue of interest (e.g., to face anteriorly when positioned at the back of the patient's neck) a second major surface 204 opposite the first, and a depth D or thickness of the housing 201 extending between the first and second major surfaces. The housing 201 can define a superior side surface 206 (e.g., configured to face superiorly when the device 110 is implanted in or at the patient's neck) and an opposing inferior side surface 208. The housing 201 can further include a central portion 205, a first lateral portion (or left portion) 207, and a second lateral portion (or right portion) 209. The electrodes 213 are distributed about the housing 201 such that a central electrode 213b is disposed within the central portion 205 (e.g., substantially centrally along a horizontal axis of the device), a left electrode 213a electrode is disposed within the left portion 207, and a right electrode 213c is disposed within the right portion 209. As illustrated, the housing 201 can define a boomerang or chevron-like shape in which the central portion 205 includes a vertex, with the first and second lateral portions 207 and 209 extending both laterally outward and from the central portion 205 and also at a downward angle with respect to a horizontal axis of the device.

The configuration of the housing 201 may facilitate placement either over the user's skin in a bandage-like form or for subcutaneous implantation. As such, a relatively thin housing 201 can be advantageous. Additionally, the housing 201 can be flexible in some examples, so that the housing 201 can at least partially bend to correspond to the anatomy of the patient's neck (e.g., with left and right lateral portions 207 and 209 of the housing 201 bending anteriorly relative to the central portion 205 of the housing 201).

In some examples, the housing 201 can have a length L of between about 15-50 mm, between about 20-30 mm, or about 25 mm. The housing 201 can have a width W of between about 2.5-15 mm, between about 5-10 mm, or about 7.5 mm. In some examples, the housing 201 can have a thickness of the thickness is less than about 10 mm, about 9 mm, about 8 mm, about 7 mm, about 6 mm, about 5 mm, about 4 mm, or about 3 mm. In some examples, the thickness of the housing 201 can be between about 2-8 mm, between 3-5 mm, or about 4 mm. The housing 201 can have a volume of less than about 1.5 cc, about 1.4 cc, about 1.3 cc, about 1.2 cc, about 1.1 cc, about 1.0 cc, about 0.9 cc, about 0.8 cc, about 0.7 cc, about 0.6 cc, about 0.5 cc, or about 0.4 cc. In some examples, the housing 201 can have dimensions suitable for implantation through a trocar introducer or any other suitable implantation technique.

As illustrated, the electrodes 213 carried by the housing 201 are arranged so that all three electrodes 213 do not lie on a common axis. In such a configuration, the electrodes 213 can achieve a better signal vector as compared to electrodes that are all aligned along a single axis. This can be particularly useful in a sensor device 210 configured to be implanted at the neck while detecting electrical activity in the brain. In some examples, this electrode configuration also provides for improved cardiac ECG sensitivity by integrating 3 potential signal vectors.

In the example shown in FIG. 6B, all three electrodes 213 are located on the first major surface 203 and are substantially flat and outwardly facing. However, in other examples one or more electrodes 213 may utilize a three-dimensional configuration (e.g., curved around an edge of the device 210). Similarly, in other examples one or more electrodes 213 may be disposed on the second major surface opposite the first. The various electrode configurations allow for configurations in which electrodes 213 are located on both the first major surface and the second major surface. In other configurations, such as that shown in FIG. 6B, electrodes 213 are only disposed on one of the major surfaces of the housing 201. The electrodes 213 may be formed of a plurality of different types of biocompatible conductive material (e.g., stainless steel, titanium, platinum, iridium, alloys thereof, or conductive polymers such as poly(3,4-ethylenedioxythiophene) polystyrene sulfonate (PEDOT)) and may utilize one or more coatings such as titanium nitride or fractal titanium nitride. In some examples, the material choice for electrodes can also include materials having a high surface area (e.g., to provide better electrode capacitance for better sensitivity) and roughness (e.g., to aid implant stability). Although the example shown in FIG. 6 includes three electrodes 213, in some examples the sensor device 210 can include 1, 2, 4, 5, 6, or more electrodes carried by the housing 201.

FIG. 6C illustrates another example in which the electrodes 213 are not exposed along the first major surface 203 of the housing 201. Instead, the electrodes 213 can be exposed along superior and inferior side surfaces (e.g., facing superiorly and inferiorly when implanted at or on a patient's neck), as shown in FIGS. 6D and 6E. FIG. 6F illustrates another example in which the housing 201 assumes a curved configuration, and in which the electrodes can be place along the superior and/or inferior side surfaces of the housing 201. In some examples, a curved configuration can improve patient comfort and more readily conform to the anatomy of the patient's neck region.

In operation, the electrodes 213 may be used to sense electrical signals (e.g., EEG signals and/or EP signals) which may be submuscular or subcutaneous. The sensed electrical signals may be stored in a memory of the sensor device 210, and signal data may be transmitted via a communications link to another device (e.g., external device 150 of FIG. 5). The sensed electrical signals may be time-coded or otherwise correlated with time data, and stored in this form, so that the recency, frequency, time of day, time span, or date(s) of a particular signal data point or data series (or computed measures or statistics based thereon) may be determined and/or reported. In some examples, electrodes 213 may additionally or alternatively be used for sensing any bio-potential signal of interest, such as EP, an ECG, intracardiac EGM, EMG, or a nerve signal, from any implanted location. These data may be time-coded or time-correlated, and stored in that form, in the manner described above with respect to EEG signal data.

FIG. 7 illustrates other examples of sensor device 310. In some examples, the sensor device 310 can include some or all of the features of the sensor devices 16, 110 and/or 210 described above with respect to FIGS. 5 and 6 in accordance with examples and can include additional features as described in connection with FIG. 7. In some examples, sensor device 310 is an example of sensor device 16 shown in FIG. 1. In the example shown in FIG. 7, sensor device 310 may be a monitoring device having housing 314, proximal electrode 313a and distal electrode 313b (individually or collectively “electrode 313” or “electrodes 313”). Housing 314 may further comprise first major surface 318, second major surface 320, proximal end 322, and distal end 324. Housing 314 encloses electronic circuitry located inside the sensor device 310 and protects the circuitry contained therein from body fluids. Electrical feedthroughs provide electrical connection of electrodes 313. In an example, sensor device 310 may be an external monitor, such as patch that may be positioned on an external surface of the patient, or another type of medical device (e.g., instead of as an ICM), such as described further herein.

In the example shown in FIG. 7, sensor device 310 is defined by a length “L,” a width “W,” and thickness or depth “D.” sensor device 310 may be in the form of an elongated rectangular prism wherein the length L is significantly larger than the width W, which in turn is larger than the depth D. In one example, the geometry of the sensor device 310—in particular, a width W being greater than the depth D—is selected to allow sensor device 310 to be inserted under the skin of the patient using a minimally invasive procedure and to remain in the desired orientation during insertion. For example, the device shown in FIG. 7 includes radial asymmetries (notably, the rectangular shape) along the longitudinal axis that maintains the device in the proper orientation following insertion. For example, in one example the spacing between proximal electrode 313a and distal electrode 313b may range from 30 millimeters (mm) to 55 mm, 35 mm to 55 mm, and from 40 mm to 55 mm and may be any range or individual spacing from 25 mm to 60 mm. In-some examples, the length L may be from 30 mm to about 70 mm. In other examples, the length L may range from 40 mm to 60 mm, 45 mm to 60 mm and may be any length or range of lengths between about 30 mm and about 70 mm. In addition, the width W of first major surface 18 may range from 3 mm to 10 mm and may be any single or range of widths between 3 mm and 10 mm. The thickness of depth D of sensor device 310 may range from 2 mm to 9 mm. In other examples, the depth D of sensor device 310 may range from 2 mm to 5 mm and may be any single or range of depths from 2 mm to 9 mm. In addition, sensor device 310 according to an example of the present disclosure is has a geometry and size designed for ease of implant and patient comfort. Examples of sensor device 310 described in this disclosure may have a volume of 3 cc or less, 2 cc or less, 1 cc or less, 0.9 cc or less, 0.8 cc or less, 0.7 cc or less, 0.6 cc or less, 0.5 cc or less, or 0.4 cc or less, any volume between 3 and 0.4 cc. In addition, in the example shown in FIG. 7, proximal end 322 and distal end 324 are rounded to reduce discomfort and irritation to surrounding tissue once inserted under the skin of the patient.

In the example shown in FIG. 7, once inserted within the patient, the first major surface 318 faces outward, toward the skin of the patient while the second major surface 320 is located opposite the first major surface 318. Consequently, the first and second major surfaces may face in directions along a sagittal axis of patient, and this orientation may be consistently achieved upon implantation due to the dimensions of sensor device 310. Additionally, an accelerometer, or axis of an accelerometer, may be oriented along the sagittal axis.

Proximal electrode 313a and distal electrode 313b are used to sense electrical signals (e.g., EEG signals and/or EP signals) which may be submuscular or subcutaneous. Electrical signals may be stored in a memory of the sensor device 310, and signal data may be transmitted via integrated antenna 326 to another medical device, which may be another implantable device or an external device, such as external device 150 (FIG. 5). In some examples, electrodes 313a and 313b may additionally or alternatively be used for sensing any bio-potential signal of interest, such as an electrocardiogram (ECG), intracardiac electrogram (EGM), electromyogram (EMG), or a nerve signal, from any implanted location.

In the example shown in FIG. 7, proximal electrode 313a is in close proximity to the proximal end 322, and distal electrode 313b is in close proximity to distal end 324. In this example, distal electrode 313b is not limited to a flattened, outward facing surface, but may extend from first major surface 318 around rounded edges 328 or end surface 330 and onto the second major surface 320 so that the electrode 313b has a three-dimensional curved configuration. In the example shown in FIG. 7, proximal electrode 313a is located on first major surface 318 and is substantially flat, outward facing. However, in other examples proximal electrode 313a may utilize the three-dimensional curved configuration of distal electrode 313b, providing a three-dimensional proximal electrode (not shown in this example). Similarly, in other examples distal electrode 313b may utilize a substantially flat, outward facing electrode located on first major surface 18 similar to that shown with respect to proximal electrode 313a. The various electrode configurations allow for configurations in which proximal electrode 313a and distal electrode 313b are located on both first major surface 18 and second major surface 320. In other configurations, such as that shown in FIG. 7, only one of proximal electrode 313a and distal electrode 313b is located on both major surfaces 318 and 320, and in still other configurations both proximal electrode 313a and distal electrode 313b are located on one of the first major surface 318 or the second major surface 320 (e.g., proximal electrode 313a located on first major surface 318 while distal electrode 313b is located on second major surface 320). In another example, sensor device 310 may include electrodes 313 on both first major surface 318 and second major surface 320 at or near the proximal and distal ends of the device, such that a total of four electrodes 313 are included on sensor device 310. Electrodes 313 may be formed of a plurality of different types of biocompatible conductive material (e.g., stainless steel, titanium, platinum, iridium, or alloys thereof), and may utilize one or more coatings such as titanium nitride or fractal titanium nitride. Although the example shown in FIG. 7 includes two electrodes 313, in some examples the sensor device 310 can include 3, 4, 5, or more electrodes carried by the housing 314 or by an electrode extension(s).

In the example shown in FIG. 7, proximal end 322 includes a header assembly 332 that includes one or more of proximal electrode 313a, integrated antenna 326, anti-migration projections 334, or suture hole 336. Integrated antenna 326 is located on the same major surface (i.e., first major surface 318) as proximal electrode 313a and is also included as part of header assembly 332. Integrated antenna 326 allows sensor device 310 to transmit or receive data. In other examples, integrated antenna 326 may be formed on the opposite major surface as proximal electrode 313a, or may be incorporated within the housing 314 of sensor device 310. In the example shown in FIG. 7, anti-migration projections 334 are located adjacent to integrated antenna 326 and protrude away from first major surface 318 to prevent longitudinal movement of the device. In the example shown in FIG. 7 anti-migration projections 334 includes a plurality (e.g., six or nine) small bumps or protrusions extending away from first major surface 318. As discussed above, in other examples anti-migration projections 334 may be located on the opposite major surface as proximal electrode 313a or integrated antenna 326. In addition, in the example shown in FIG. 4 header assembly 332 includes suture hole 336, which provides another means of securing sensor device 310 to the patient to prevent movement following insert. In the example shown, suture hole 336 is located adjacent to proximal electrode 313a. In one example, header assembly 332 is a molded header assembly made from a polymeric or plastic material, which may be integrated or separable from the main portion of sensor device 310.

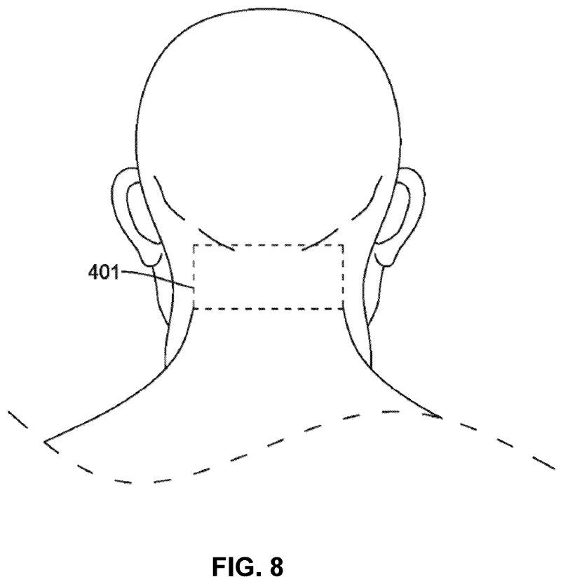

FIG. 8 illustrates an exemplary target region 401 for positioning a sensor device (e.g., sensor devices 16, 110, 210, 310 described elsewhere herein). As illustrated, the target region 401 can be a rear portion of a user's neck or at the base of the skull. The target region 401 can be positioned above the patient's shoulders and at or below the patient's occipital bone. As noted previously, a sensor device can be disposed in this region either via implantation (e.g., subcutaneously) or by being placed over the patient's skin with one or more electrodes of a sensor device being in direct contact with the patient's skin at or adjacent the target region 401.

While conventional EEG electrodes are placed over the patient's scalp, the present technology advantageously enables recording of clinically useful brain activity data via electrodes positioned at the target region 401 at the rear of the patient's neck. This anatomical area is well suited to suited both to implantation of a sensor device and to temporary placement of a sensor device over the patient's skin. In contrast, EEG electrodes positioned over the scalp are cumbersome, and implantation over the patient's skull is challenging and may introduce significant patient discomfort. As noted elsewhere here, conventional EEG electrodes are typically positioned over the scalp to more readily achieve a suitable signal-to-noise ratio for detection of brain activity. However, by using certain digital signal processing, and a special-purpose classifier algorithm, clinically useful brain activity data can be obtained using sensors disposed at the target region 401. Specifically, the electrodes can detect electrical activity that corresponds to brain activity in the P3, Pz, and/or P4 regions (see FIG. 9).

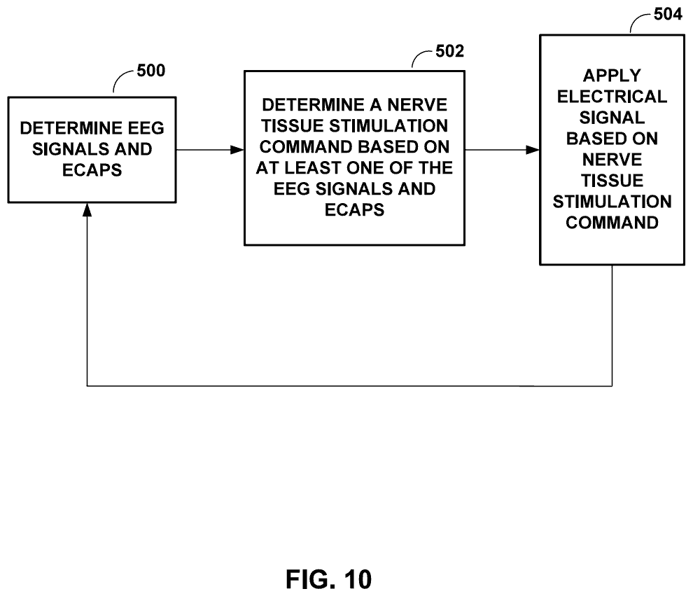

FIG. 10 is a flow diagram illustrating example techniques according to the present disclosure. Sensor device 16 may sense EEG signals of patient 14 and IMD 28 may determine ECAPs (500). Sensor device 16 may transmit the sensed EEG signals to computing device 12. IMD 28 may transmit ECAP(s) to computing device 12. In some examples, IMD 28 may apply an initial electrical signal to provide an initial stimulation to the nerve tissue and sense the ECAPs in response to the initial stimulation. Circuitry of computing device 12, sensor device 16, and/or IMD 28 may determine a nerve tissue stimulation command based on at least one of the EEG signals and the ECAP(s) (502). Computing device 12 may transmit the nerve tissue stimulation command to IMD 28. In some examples, circuitry of computing device 12, sensor device 16, and/or IMD 28 may determine an indication of a cranial acute health event based on EEG signals and determine the nerve tissue stimulation command based on the indication of the cranial acute health event and/or the ECAPs. In some examples, circuitry of computing device 12, sensor device 16, and/or IMD 28 may determine an indication of a cranial acute health event based on EEG signals, determine one or more features of the cranial acute health event based on the EEG signals, and determine the nerve tissue stimulation command based on the one or more features of the cranial acute health event and/or the ECAPs. In some examples, the nerve tissue includes vagus nerve tissue.

IMD 28 may apply an electrical signal to stimulate nerve tissue baes on the received nerve tissue command (504). In some examples, a nerve tissue stimulation command includes one or more of protocols, parameters, or regimes of electrical signal to be applied to stimulate nerve tissue. In some examples, the nerve tissue stimulation command includes one or more of an amplitude or frequency of the electrical signal to be applied. In some examples, the IMD 28 is configured to apply the electrical signal to stimulate nerve tissue via at least one of plurality of electrodes of lead 112. In some examples, IMD 28 is configured to sense the ECAPs via at least one of plurality of electrodes of lead 112. In some examples, after IMD 28 applies an electrical signal, the techniques return to (500) where sensor device 16 senses additional EEG signals of patient and IMD determines additional ECAPs of patient.

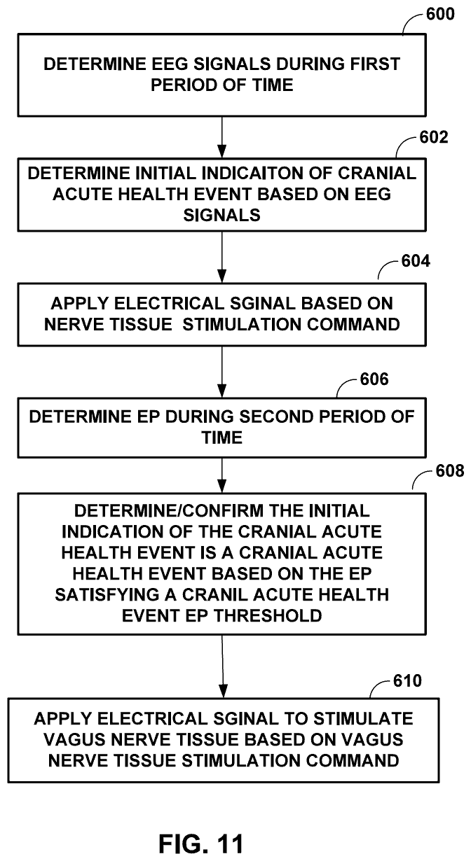

FIG. 11 is a flow diagram illustrating example techniques according to the present disclosure. Sensor device 16 may sense EEG signals of patient 14 during a first period of time (600). In some examples, sensor device 16 may transmit the sensed EEG signals to computing device 12. Circuitry of computing device 12, sensor device 16, and/or IMD 28 may determine an initial indication of a cranial acute health event based on the EEG signals (602). Circuitry of computing device 12, sensor device 16, and/or IMD 28 determine a nerve tissue stimulation command based on a determination of the initial indication of a cranial acute health event. In some examples computing device 12 and/or sensor device 16 may transmit a nerve tissue stimulation command to IMD 28 in response to a determination of the initial indication of a cranial acute health event. IMD 28 may receive the nerve tissue stimulation command and apply electrical signal(s) to stimulate nerve tissue based on the received nerve tissue command (604). In some examples, nerve tissue stimulation command includes one or more of an amplitude or frequency of the electrical signal to be applied. In some examples, the nerve tissue may include median nerve tissue. Sensor device 16 may sense an EP signal during a second period of time and determine an EP based on the sensed EP signal (606). The second period of time is after the first period of time and after the second IMD applies an electrical signal to stimulate nerve tissue, such as median nerve tissue. Sensor device 16 may transmit the EP to computing device 12 and computing device 12 may receive the EP from sensor device 16. Circuitry of computing device 12, sensor device 16, and/or IMD 28 may determine/confirm the initial indication is a cranial acute health event based on the EP satisfying a cranial acute health event EP threshold (608). In some examples, the EP satisfies the cranial acute health event EP threshold when a value of the EP is less than 50% of the cranial acute health event EP threshold.

In some examples, in response to a determination that the initial indication of the cranial acute health event is a cranial acute health event, computing device 12 and/or sensor device 16 may transmit a vagus nerve tissue stimulation command to IMD 28 and IMD 28 may receive the vagus nerve tissue stimulation command from computing device 12 and/or sensor device 16. IMD 28 may apply an electrical signal to stimulate vagus nerve tissue based on the received vagus nerve tissue stimulation command (610).

The techniques of this disclosure may facilitate closed-loop stimulation of nerve tissue, such as the vagus nerve and/or median nerves, in a manner that may provide quicker, more efficient, and more responsive results to cranial acute health events than an open-loop technique.

It should be noted that the techniques described herein, may not be limited to treatment or monitoring of a human patient. In alternative examples, the techniques of this disclosure may be applied to non-human patients, e.g., primates, canines, equines, pigs, and felines. These other animals may undergo clinical or research therapies that my benefit from the subject matter of this disclosure.

Various examples are discussed relative to one or more stimulation devices. It is recognized that the stimulation devices may include features and functionality in addition to electrical stimulation. Many of these additional features are expressly discussed herein. A few example features include, but are not limited to, different types of sensing capabilities and different types of wireless communication capabilities. For case of discussion, the present disclosure does not expressly recite every conceivable combination of the additional features, such as by repeating every feature each time different examples and uses of the stimulation devices are discussed.

The techniques of this disclosure may be implemented in a wide variety of computing devices, medical devices, or any combination thereof. Any of the described units, circuitry or components may be implemented together or separately as discrete but interoperable logic devices. Depiction of different features as circuitry or units is intended to highlight different functional aspects and does not necessarily imply that such circuitry or units must be realized by separate hardware or software components. Rather, functionality associated with one or more circuitry or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various circuitry and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processing circuitry, as well as any combinations of such components, remote servers, remote client devices, or other devices. The term “processing circuitry” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, circuitry or components may be implemented together or separately as discrete but interoperable logic devices. Depiction of different features as circuitry or units is intended to highlight different functional aspects and does not necessarily imply that such circuitry or units must be realized by separate hardware or software components. Rather, functionality associated with one or more circuitry or units may be performed by separate hardware or software components, or integrated within common or separate hardware or software components. For example, any circuitry described herein may include electrical circuitry configured to perform the features attributed to that particular circuitry, such as fixed function processing circuitry, programmable processing circuitry, or combinations thereof.

In some examples, a computer-readable storage medium comprises non-transitory medium. The term “non-transitory” may indicate that the storage medium is not embodied in a carrier wave or a propagated signal. In certain examples, a non-transitory storage medium may store data that may, over time, change (e.g., in RAM or cache).

This disclosure includes the following non-limiting examples.

Example 1: A system includes a first implantable medical device (IMD) configured to sense electroencephalogram (EEG) signals of a patient; a second IMD configured to determine evoked compound action potential (ECAP) of the patient; and processing circuitry of at least one of the first IMD, the second IMD, or a computing device configured for wireless communication with at least one of the first IMD or the second IMD, the processing circuitry configured to determine a nerve tissue stimulation command based on at least one of the EEG signals and the ECAP, wherein the second IMD is further configured to apply an electrical signal to stimulate nerve tissue based on the nerve tissue stimulation command.

Example 2: The system of example 1, wherein the second IMD is further configured to: apply an initial electrical signal to provide an initial stimulation to the nerve tissue; and sense the ECAP in response to the initial stimulation.

Example 3: The system of any of examples 1-2, wherein to determine the nerve tissue stimulation command, the processing circuitry is further configured to: determine an indication of a cranial acute health event based on the EEG signals; and determine the stimulation command based on the indication of the cranial acute health event and the ECAP.

Example 4: The system of any of examples 1-2, wherein to determine the nerve tissue stimulation command, the processing circuitry is further configured to: determine an indication of a cranial acute health event based on the EEG signals; determine one or more features of the cranial acute health event based on the EEG signals; and determine the stimulation command based on the one or more features of the cranial acute health event and the ECAP.

Example 5: The system of any of examples 1-4, wherein the second IMD is configured to sense ECAP of the nerve tissue.

Example 6: The system of any of examples 1-5, wherein the nerve tissue includes vagus nerve tissue.

Example 7: The system of any of examples 1-6, wherein the nerve tissue stimulation command includes one or more of an amplitude or frequency of the electrical signal to be applied.

Example 8: The system of any of examples 1-7, wherein the first IMD is configured to be implanted on a head of the patient.

Example 9: The system of any of examples 1-8, further comprising a lead coupled to the second IMD, the lead including a plurality of electrodes, wherein the second IMD is configured to apply the electrical signal to stimulate nerve tissue via at least one of plurality of electrodes of the lead.

Example 10: The system of example 9, wherein the lead is configured to sense ECAP signals via at least one of the plurality of electrodes of the lead and the second IMD is configured to determine the ECAP based on the sensed ECAP signals.

Example 11: The system of any of examples 1-10, wherein the processing circuitry is positioned in the computing device, the first IMD is configured to transmit the sensed EEG signals to the computing device, the second IMD is configured to transmit the sensed ECAP to the computing device, and the computing device is configured to transmit the nerve tissue stimulation command to the second IMD.

Example 12: A system includes a first implantable medical device (IMD) configured to sense electroencephalogram (EEG) signals of a patient and sense an evoked potential (EP) signal of the patient; a second IMD configured to an apply electrical signal to stimulate nerve tissue; and processing circuitry of at least one of the first IMD, the second IMD, or a computing device configured for wireless communication with at least one of the first IMD or the second IDM, the processing circuitry configured to: determine an initial indication of a cranial acute health event based on the EEG signals during the first period of time; and determine a nerve tissue stimulation command based on the determination of the initial indication of the cranial acute health event, wherein the second IMD is further configured to apply an electrical signal to stimulate nerve tissue based on the nerve tissue stimulation command, and wherein the processing circuitry is further configured to determine an EP occurring during a second period of time based on the sensed EP signal, the second period of time being after the first period of time and after the second IMD applies an electrical signal to stimulate nerve tissue, and determine the initial indication of the cranial acute health event is a cranial acute health event based, at least, on the EP satisfying a cranial acute health event EP threshold.

Example 13: The system of example 12, wherein the processing circuitry is positioned in the computing device, the first IMD is configured to transmit the sensed EEG signals and the sensed EP signal to the computing device, and the processing circuitry is configured to transmit the nerve tissue stimulation command to the second IMD.

Example 14: The system of any of examples 12-13, wherein the EP satisfies the cranial acute health event EP threshold when a value of the EP is less than 50% of the cranial acute health event EP threshold.

Example 15: The system of any of examples 12-14, wherein the nerve tissue includes median nerve tissue.

Example 16: The system of any of examples 12-15, wherein the nerve tissue stimulation command includes one or more of an amplitude or frequency of the electrical signal to be applied.

Example 17: The system of any of examples 12-16, wherein the nerve tissue stimulation command comprises a median nerve tissue stimulating command, and the processing circuitry is further configured to: determine a vagus nerve tissue stimulation command based on the determination that the initial indication of the cranial acute health event is a cranial acute health event.

Example 18: The system of example 17, wherein the second IMD is further configured to: apply an electrical signal to stimulate vagus nerve tissue based on the vagus nerve tissue stimulation command.

Example 19: The system of any of examples 11-18, wherein the first IMD is configured to be implanted on a head of the patient.

Example 20: The system of any of examples 11-19, further comprising a lead coupled to the second IMD, the lead including a plurality of electrodes, wherein the second IMD is configured to apply the electrical signal to stimulate nerve tissue via at least one of the plurality of electrodes of the lead.

Various examples have been described herein. Any combination of the described operations or functions is contemplated. These and other examples are within the scope of the following claims. Based upon the above discussion and illustrations, it is recognized that various modifications and changes may be made to the disclosed examples in a manner that does not require strictly adherence to the examples and applications illustrated and described herein. Such modifications do not depart from the true spirit and scope of various aspects of the disclosure, including aspects set forth in the claims.

What is claimed is:

1. A system comprising:
a first implantable medical device (IMD) configured to sense electroencephalogram (EEG) signals of a patient;
a second IMD configured to determine evoked compound action potential (ECAP) of the patient; and
processing circuitry of at least one of the first IMD, the second IMD, or a computing device configured for wireless communication with at least one of the first IMD or the second IMD, the processing circuitry configured to determine a nerve tissue stimulation command based on at least one of the EEG signals and the ECAP,
wherein the second IMD is further configured to apply an electrical signal to stimulate nerve tissue based on the nerve tissue stimulation command.

2. The system of claim 1, wherein the second IMD is further configured to:
apply an initial electrical signal to provide an initial stimulation to the nerve tissue; and
sense the ECAP in response to the initial stimulation.

3. The system of claim 1, wherein to determine the nerve tissue stimulation command, the processing circuitry is further configured to:
determine an indication of a cranial acute health event based on the EEG signals; and
determine the stimulation command based on the indication of the cranial acute health event and the ECAP.

4. The system of claim 1, wherein to determine the nerve tissue stimulation command, the processing circuitry is further configured to:
determine an indication of a cranial acute health event based on the EEG signals;
determine one or more features of the cranial acute health event based on the EEG signals; and
determine the stimulation command based on the one or more features of the cranial acute health event and the ECAP.

5. The system of claim 1, wherein the second IMD is configured to sense ECAP of the nerve tissue.

6. The system of claim 1, wherein the nerve tissue includes vagus nerve tissue.

7. The system of claim 1, wherein the nerve tissue stimulation command includes one or more of an amplitude or frequency of the electrical signal to be applied.

8. The system of claim 1, wherein the first IMD is configured to be implanted on a head of the patient.

9. The system of claim 1, further comprising a lead coupled to the second IMD, the lead including a plurality of electrodes,
wherein the second IMD is configured to apply the electrical signal to stimulate nerve tissue via at least one of plurality of electrodes of the lead.

10. The system of claim 9, wherein the lead is configured to sense ECAP signals via at least one of the plurality of electrodes of the lead and the second IMD is configured to determine the ECAP based on the sensed ECAP signals.

11. The system of claim 1, wherein the processing circuitry is positioned in the computing device,
the first IMD is configured to transmit the sensed EEG signals to the computing device,
the second IMD is configured to transmit the sensed ECAP to the computing device, and
the computing device is configured to transmit the nerve tissue stimulation command to the second IMD.

12. A system comprising:
a first implantable medical device (IMD) configured to sense electroencephalogram (EEG) signals of a patient and sense an evoked potential (EP) signal of the patient;
a second IMD configured to an apply electrical signal to stimulate nerve tissue; and
processing circuitry of at least one of the first IMD, the second IMD, or a computing device configured for wireless communication with at least one of the first IMD or the second IDM, the processing circuitry configured to:
determine an initial indication of a cranial acute health event based on the EEG signals during the first period of time; and
determine a nerve tissue stimulation command based on the determination of the initial indication of the cranial acute health event,
wherein the second IMD is further configured to apply an electrical signal to stimulate nerve tissue based on the nerve tissue stimulation command, and
wherein the processing circuitry is further configured to determine an EP occurring during a second period of time based on the sensed EP signal, the second period of time being after the first period of time and after the second IMD applies an electrical signal to stimulate nerve tissue, and determine the initial indication of the cranial acute health event is a cranial acute health event based, at least, on the EP satisfying a cranial acute health event EP threshold.

13. The system of claim 12, wherein the processing circuitry is positioned in the computing device,
the first IMD is configured to transmit the sensed EEG signals and the sensed EP signal to the computing device, and
the processing circuitry is configured to transmit the nerve tissue stimulation command to the second IMD.

14. The system of claim 13, wherein the EP satisfies the cranial acute health event EP threshold when a value of the EP is less than 50% of the cranial acute health event EP threshold.

15. The system of claim 14, wherein the nerve tissue includes median nerve tissue.

16. The system of claim 15, wherein the nerve tissue stimulation command includes one or more of an amplitude or frequency of the electrical signal to be applied.

17. The system of claim 16, wherein the nerve tissue stimulation command comprises a median nerve tissue stimulating command, and the processing circuitry is further configured to:
determine a vagus nerve tissue stimulation command based on the determination that the initial indication of the cranial acute health event is a cranial acute health event.

18. The system of claim 17, wherein the second IMD is further configured to:
apply an electrical signal to stimulate vagus nerve tissue based on the vagus nerve tissue stimulation command.

19. The system of claim 18, wherein the first IMD is configured to be implanted on a head of the patient.

20. The system of claim 19, further comprising a lead coupled to the second IMD, the lead including a plurality of electrodes,
wherein the second IMD is configured to apply the electrical signal to stimulate nerve tissue via at least one of the plurality of electrodes of the lead.
